(19) United States
(12) Patent Application Publication
(10) Pub. No.: US 2015/0224094 A1
(43) Pub. Date: Aug. 13, 2015

(54) ADMINISTRATION OF ACETYLCHOLINESTERASE INHIBITORS TO MITIGATE NEUROTOXIN-INDUCED PARALYSIS AND RESIDUAL NEUROMUSCULAR BLOCKADE

(71) Applicant: Matthew R. LEWIN, (US)

(72) Inventor: Matthew R. Lewin, Corte Madera, CA (US)

(73) Assignee: OPHIREX, INC., Corte Madera, CA (US)

(21) Appl. No.: 14/427,035

(22) PCT Filed: Sep. 6, 2013
(86) PCT No.: PCT/US2013/058640

§ 371 Date: Mar. 10, 2015

Related U.S. Application Data
(60) Provisional application No. 61/743,705, filed on Sep. 10, 2012.
(60) Provisional application No. 61/771,750, filed on Mar. 1, 2013.
(60) Provisional application No. 61/824,087, filed on May 16, 2013.
(60) Provisional application No. 61/857,032, filed on Jul. 22, 2013.

(51) Int. Cl. A61K 31/4425; A61K 9/00; A61K 31/40

(52) U.S. Cl. CPC A61K 31/4425; A61K 31/40; A61K 9/0043

(57) ABSTRACT

Methods and kits for treating or reducing the likelihood of neurotoxin-induced respiratory failure in a subject, such as a victim of neurotoxic envenomation are provided. Also provided are methods for treating or reducing the likelihood of residual neuromuscular blockade in a subject to whom a nondepolarizing neuromuscular blocking agent has been administered. The methods involve administering a pharmaceutically effective dose of an acetylcholinesterase inhibitor to the subject, where the administration is not via injection. In some embodiments intra-nasal or ocular administration is used.

### CROSS-REFERENCES TO RELATED APPLICATIONS

This application claims priority to provisional application No. 61/743,705, filed Sep. 10, 2012; provisional application No. 61/771,750, filed Mar. 1, 2013; provisional application No. 61/824,087, filed May 16, 2013; and provisional application No. 61/857,032, filed Jul. 22, 2013. The entire content of each of the aforementioned applications is incorporated herein by reference.

### FIELD OF THE INVENTION

The present invention relates to kits and treatment methods for neurotoxin-induced paralysis and respiratory failure.

### BACKGROUND OF THE INVENTION

Neurotoxins are compounds that inhibit the ability of a neuron to control its ion channels or interfere with communication between neurons across a synapse. A well-known class of neurotoxins are peptides contained in venom. These are toxins used by an animal to immobilize prey or to defend itself. Venom is generally delivered to a victim by bite or insertion of a sharp body feature. Although many venoms cause only discomfort, some venoms are highly poisonous and can result in a victim's death. Examples of venomous animals include invertebrates (e.g., black widow spiders, box jellyfish, and cone snails); fish (e.g., puffer fish or other members of the family Tetraodontidae) and reptiles (e.g., snakes and beaded lizards).

Notably, bites from venomous snakes result in is a major public health problem in many countries and on all continents except Antarctica. It is estimated that, worldwide, there may be more than five million instances of snake bite per year, out of which approximately 400,000 result in severe sequelae including as many as 125,000 deaths and many thousands more with permanent disability. It is estimated that over 40,000 snake bite victims, mostly young, die each year in India. In the U.S., about 7,000 poisonous snake bites occur each year, and more than 1,500 snakebites per year in Australia are from purely neurotoxic snake envenomations. See Alirol et al., 2010, “Snake Bite in South Asia: A Review” PLoS Negl Trop Dis. 4(1): e603; and Kasturiratne et al., 2008, “The global burden of snakebite: a literature analysis and modeling based on regional estimates of envenoming and deaths,” PLoS Med. 5:e218.

Many of the poisonous venoms are acetylcholine-mediated neurotoxins (ACh-mediated neurotoxin) which paralyze skeletal muscles. Although anti-venoms are sometimes available, their value in treating victims is limited for a variety of reasons. First, the animal must be identified so the appropriate anti-venom can be used. Second, even the correct identification of the animal does not guarantee that an anti-venom has been developed. Third, and perhaps most importantly, even if the venom has been identified and a corresponding anti-venom exists, the likelihood that the victim has ready access to the anti-venom is exceedingly low. Most anti-venoms are readily perishable and not generally available outside of a hospital setting. Moreover, because venomous bites often occur in remote locations far from population centers, the victim is not likely to be able to reach a hospital in time to receive the needed treatment.

In addition to antivenom, complementary approaches have been used to manage snake bite. The acetylcholinesterase inhibitor edrophonium has been administered intravenously for management of snake bite (Warrell et al., 1983, “Severe neurotoxic envenoming by the Malayan krait Bungarus candidus (Linnaeus): response to antivenom and anticholinesterase,” Br Med J (Clin Res Ed) 286(6366):678-80; Watt et al., 1986, “Positive response to edrophonium in patients with neurotoxic envenoming by cobras (Naja naja philippinensis). A placebo-controlled study” N Engl J Med. 315(23):1444-8; and Currie et al., 1988, “Resolution of neurotoxicity with anticholinesterase therapy in death-adder envenomation. Med. J. Aust. 148:522-525). Use of certain acetylcholinesterase inhibitors has been proposed for treatment of Myasthenia gravis, Alzheimer's Disease, and glaucoma (see Walker, 1935, “Case showing the Effect of Prostigmin on Myasthenia Gravis.” Proc R Soc Med 28: 759-61; Satyamurti et al., 1975, “Blockade of acetylcholine receptors: a model of myasthenia gravis.” Science 187(4180): 955-7; Costantino et al., 2008, “Intranasal administration of acetylcholinesterase inhibitors,” BMC Neuroscience 2008, 9(Suppl 3):S6; Gore et al., 1998, “Comparative biomembrane permeation of tacrine using Yucatan minipigs and domestic pigs as the animal model” J Pharm Sci 87:441-447; Hussain et al., 1991, “Intranasal absorption of physostigmine and arecoline” J Pharm Sci 1991, 80:750-751; Dahlin et al., 2001, “Nasal administration of a physostigmine analogue (NXX-066) for Alzheimer's disease to rats,” Int J Pharm 212:267-274; Deroetth et al., 1965, Effect of Phospholine Iodide on Blood Cholinesterase Levels of Normal and Glaucoma Subjects. Am J Ophthalmol 59: 586-592; De Roetth et al., 1966, “Blood cholinesterase activity of glaucoma patients treated with phospholine iodide,” Am J Ophthalmol 62; Pohanka, M., 2012, Expert Opin. Ther. Patents 22(8), incorporated herein by reference).

There is a clear unmet need for effective and economical methods for treatment of acute neurotoxic envenomation.

### BRIEF SUMMARY OF THE INVENTION

In one aspect, the invention relates to administration of an acetylcholinesterase inhibitor to a subject to treat, or reduce the likelihood of development of, neurotoxin-induced respiratory failure, wherein the inhibitor is not administered by injection. In some embodiments intranasal administration (e.g., resulting in drug uptake through the nasal epithelium and/or lungs) or a mask, nebulizer, metered dose inhaler, spray device, or gel, or administration via the eye (e.g., ophthalmic drops or ophthalmic ointment) is used.

In some embodiments, the subject is at risk of neurotoxin-induced respiratory failure due to a snake or insect bite or sting.

In some embodiments, the subject is at risk of neurotoxin-induced respiratory failure due to envenomation by a snake, arthropod, mollusk or cnidarian.

In one aspect, the invention provides a method for treating or reducing the likelihood of neurotoxin-induced respiratory failure in a human subject, comprising determining that the subject is a victim of a snake bite and administering a pharmaceutically effective dose of an acetylcholinesterase inhibitor to the subject, wherein the inhibitor is not administered by injection.

In another aspect, the invention relates to administration of an acetylcholinesterase inhibitor to reverse neuromuscular blockade (e.g., residual neuromuscular blockade) in a subject who has received clinical anesthesia, wherein the inhibitor is not administered by injection.

In one aspect, the invention provides a method for treating or reducing the likelihood of residual or persistent neuromuscular blockade in a subject to whom a nondepolarizing neuromuscular blocking agent has been administered (e.g., in a perioperative, intensive care, military or air ambulance evacuation or emergency department setting), the method comprising administering a pharmaceutically effective dose of an acetylcholinesterase inhibitor to the subject, wherein the inhibitor is not administered by injection.

In some embodiments of these inventions, the acetylcholinesterase inhibitor is ambenonium, demarcarium, donepezil, edrophonium, galantamine, huperzine A, ladostigil, lactucopicrin, neostigmine, physostigmine, pyridostigmine, rivastigmine, tacrine; phospholine iodide, ungeremamine and rx72607. In some embodiments the acetylcholinesterase inhibitor is other than neostigmine. In some embodiments the acetylcholinesterase inhibitor is irreversible or quasi-irreversible. In some embodiments the inhibitor is an organophosphorous acetylcholinesterase inhibitor such as malathion.

In some embodiments a mAChR antagonist is not administered to the subject as part of the course of treatment.

In one aspect the invention provides a kit comprising an acetycholinesterase inhibitor, an intra-nasal drug delivery device, and optionally a mAChR antagonist, for use in treating snake bite or other envenomation. In exemplary embodiments the intra-nasal drug delivery device is a mask, nebulizer, metered dose inhaler, spray device, or gel. In one aspect the invention provides a kit comprising an acetycholinesterase inhibitor, an ocular drug delivery device, and optionally a mAChR antagonist. In an exemplary embodiment the ocular drug delivery device is an eye dropper. The inhibitor may be in the form of a solution, powder, liposomes, ointment, aerosol or conjugated to another compound for specific targeting and the like.

### BRIEF DESCRIPTION OF THE DRAWINGS

FIG. 1 is a schematic illustration of a neuromuscular junction.

FIG. 2 shows reversal of experimental paralysis in a human by intranasal neostigmine aerosol. Clinical measures of muscle function are represented as a function of time with baseline measurements at Time 0 at the start of mivacurium infusion and ending at 135 min with the termination of the mivacurium infusion. Intranasal neostigmine was administered at 115 min after establishing the presence of clinically significant neuromuscular impairment and electrophysiologically stable neuromuscular blockade. Stable impairment and the constant mivacurium infusion rate allowed for pre- and postneostigmine administration comparisons as illustrated by: A) progressive loss and recovery of visual acuity and B) ease of swallowing were affected before late loss of C) neck flexion, and finally, D) decrement in peak flow, followed by almost complete recovery prior to terminating mivacurium after 135 min.

FIG. 3 shows a single dose of intranasal (IN) neostigmine successfully treated 10 of 15 mice given high dose N. naja venom. (A) Venom alone (control); (B) Venom+IN neostigmine treatment. Time to euthanasia for controls (venom alone, N=5) was 193 minutes (95% CI: 36-349). 5 of 5 controls died while 10 of 15 animals treated with IN neostigmine (B) survived and were completely normal by 6 hours. Treatment with IN neostigmine (N=15) provided a significant increase in time to death or euthanasia 553 minutes (95% CI: 415-689).

### DETAILED DESCRIPTION OF THE INVENTION

In one aspect, the invention provides a method for treating or reducing the likelihood of neurotoxin-induced respiratory failure in a subject by determining that the subject is a victim of envenomation by an animal, and administering a pharmaceutically effective dose of an acetylcholinesterase inhibitor to the subject, wherein the inhibitor is not administered by injection. In will be recognized that “administered by injection” includes intravenous (IV) administration such as infusion through a catheter. Typically the inhibitor is administered via the nose (“intra-nasal administration”) or the eye (“ocular administration”). In some embodiments the animal is a snake. In some embodiments the animal is a Coral snake. In some embodiments the animal is another venomous vertebrate or an invertebrate animal. In some embodiments the subject is a human. In an embodiment, the invention provides a method for treating or reducing the likelihood of neurotoxin-induced respiratory failure in a human subject, comprising determining that the subject is a victim of a snake bite and administering a pharmaceutically effective dose of an acetylcholinesterase inhibitor to the subject, wherein the inhibitor is not administered by injection.

In some embodiments the subject treated with an acetylcholinesterase inhibitor is also treated with an mAChR antagonist. In other embodiments the subject treated with an acetylcholinesterase inhibitor with no administration of an mAChR antagonist as part of the treatment regimen.

In some embodiments the subject treated with an acetylcholinesterase inhibitor is also treated with an oxime derived acetylcholinesterase reactivating agent, such as pralidoxime, with or without a mAChR antagonist. Reactivating agents are known in the art. See, e.g., Luo et al., 2007, “An in vitro comparative study on the reactivation of nerve agent-inhibited guinea pig and human acetylcholinesterases by oximes,” Biochemistry 23; 46(42):11771-9.

In another aspect, the invention provides a method for treating or reducing the likelihood of residual neuromuscular blockade in a subject to whom a nondepolarizing neuromuscular blocking agent has been administered. The method includes administering a pharmaceutically effective dose of an acetylcholinesterase inhibitor to the subject, wherein the inhibitor is not administered by injection. In some embodiments the acetylcholinesterase inhibitor is administered intranasally, by mask or in-line with standard oxygen tubing nebulization chambers and aerosol masks, with or without mechanical ventilation. Methods for delivery of aerosols are known in the art. See, e.g., Berlinski et al., 2013, “Albuterol delivery by 4 different nebulizers placed in 4 different positions in a pediatric ventilator in vitro model.” Respir Care. 58(7):1124-33.

In another aspect, the invention provides a method for reducing the need for intensive care and emergency services by decreasing the recovery time and decreasing the duration of mechanical ventilation or other assisted breathing or for emergency reversal of nondepolarizing neuromuscular blocking agents (NMBAs) as a result of unexpected events such as inability to establish a secure airway after paralysis by an NMBA.

Without intending to be bound by a particular mechanism, it is believed that topical (e.g., intranasal, ocular, transmucosal) administration of AChl has an immediate effect based on local or regional activation of neuromuscular function or non-neuronal stores of acetylcholine in the upper airways in individuals suffering toxin-induced paralysis in advance of systemic effects related to absorption into the blood. See Example 3 describing immediate effect on facial and lingual muscles following administration. The local effect is believed, in part, to make the topical administration of the invention (e.g., via nasal spray or eye drops safer than IV formulations. Also see Brimijoin et al., 1978, “On the origin and fate of external acetylcholinesterase in peripheral nerve,” J Physiol. 285:143-58; Nguyen et al., 2000, “Choline acetyltransferase, acetylcholinesterase, and nicotinic acetylcholine receptors of human gingival and esophageal epithelia,” J Dent Res. 79(4):939-49; and Broggini et al., 1991, “Bioavailability of intranasal neostigmine: comparison with intravenous route” Methods Find Exp Clin Pharmacol. 13:193-8.

### 1. Definitions

1.1 As used herein, except where otherwise apparent from context, “snake bite” includes “dry” snake bites as well as bites that result in envenomation.

1.2 As used herein, “venom” has its normal meaning and is a poisonous secretion of an animal, such as a snake, spider, scorpion, or cone snail transmitted by a bite or sting.

1.3 As used herein, “envenomation,” refers to snake bite envenomation, i.e., injection of venom by a snake, and includes neurotoxic, non-neurotoxic envenomation, and envenomations of undetermined character. Examples of non-neurotoxic envenomation include hemotoxic, vasculotoxic, cardiotoxic, and myotoxic envenomation.

1.4 As used herein, “neurotoxic envenomation,” refers to envenomation with a neurotoxic venom. Neurotoxic venoms include, for example and not limitation, venoms produced by venoms snakes.

1.5 As used herein, a “neurotoxic venomous snake” refers to a snake having venom comprising a neurotoxin. For example and without limitation venomous snakes include Cobra, Krait, Russell's Viper, Mambas, Australian Taipan, New Guinea Death Adder, Southern Rattle Snakes, Coral snakes and sea snakes (Hydrophiinae). It will be appreciated that venoms comprise complex mixtures of proteins and other substances with toxic properties. Thus venom of a neurotoxic venomous snake may comprise agents with hemotoxic, vasculotoxic, cardiotoxic, myotoxic and/or other toxic properties, as well as neurotoxins.

1.6 As used herein, a “pharmaceutically effective dose” refers to an amount of an acetylcholinesterase inhibitor that when administered results in a clinically detectable reversal of paralysis (induced by toxin or anesthesia). An “an intra-nasal effective dose,” refers to an amount of an acetylcholinesterase inhibitor that when administered intra-nasally or via facemask results in a clinically detectable reversal of paralysis. An “an ocular effective dose,” refers to an amount of an acetylcholinesterase inhibitor that when administered via the eye results in a clinically detectable reversal of paralysis. It will be appreciated that the dose may vary somewhat with when different formulations are used. For example, a lower dose may be administered when the formulation includes a permeability enhancer.

1.7 As used herein, “intranasal administration” refers to administration into the nose. Non-limiting examples of intranasal administration include introduction of a solution or suspension in the form of a nasal spray or drops (direct instillation), intranasal application of gel, emulsion, ointment, or inhalation using, e.g., a nebulizer. See Costantino et al, “Intranasal administration of acetylcholinesterase inhibitors” BMC Neuroscience 2008, 9(Suppl 3):56. In one aspect, intranasal administration can be accomplished using a mask (e.g., nasal mask) or tube delivering an agent to the nose. This is particularly useful in the context of mitigating neuromuscular blockade. Methods of delivering agents via the nose are well known in the medical arts. Advantageously, in the context of mitigating neuromuscular blockade following anesthesia, an agent (e.g., anticholinesterase inhibitor) can be administered in-line using the same mask or apparatus used for administration of anesthesia. In some aspects, intranasal administration is accomplished nasal-oral combined exposure, with or without exposure of the eyes, e.g., using a full face mask. See, e.g., Dooley et al., 1986, “Topical therapy for oropharyngeal symptoms of myasthenia gravis.” Ann Neurol. 19(2):192-4. It will be recognized that, the route of delivery using a nasal spray or nasal drops (e.g., using a dropper or atomizer), gel, emulsion, ointment, is primarily through the nasal mucosa (nasal mucosal administration) while the route of delivery using a mask is adsorption through the respiratory tract mucosal including the lungs (i.e., via nasal mucosa, hypopharynx, and large and small airway structures). Aerosol may be delivered through endotracheal tube (see, e.g., Berlinski et al., supra).

1.8 As used herein, “ocular administration” refers to topical administration to the eye, without injection. Non-limiting examples of ocular administration include introduction of solution (eye drops), gels, ointments, and colloidal dosage forms (nanoparticles, nanomicelles, liposomes, and microemulsions). Ocular administration is well known in the art (see, e.g., Gaudana et al., 2010, “Ocular Drug Delivery” AAPS J. 12(3): 348-360, incorporated by references herein).

1.9 A “subject” as used herein, is a mammal. Generally the subject is human. In some embodiments the subject is a model experimental organism, such as mouse, rat, rabbit, pig, dog, non-human primate. In some embodiments the subject is a farm animal or pet.

1.10 As used herein, “neuromuscular blockade” means blockade resulting from administration of nondepolarizing neuromuscular blocking agents (NMBAs). Nondepolarizing NMBAs compete with acetylcholine to bind to postsynaptic nicotinic receptors.

1.11 As used herein, “residual neuromuscular blockade (RNMB)” refers to residual effects after clinical anesthesia. RNMB is described in Wilson et al., Crit Care Nurse June 2012 Vol. 32 No. 3 e1-e10, and in Kopman et al., Anesthesiology September 2008 Vol. 109 No. 3, pp 363-364, both incorporated herein by reference.

1.12 As used herein, a “therapeutically effective amount” or “therapeutically effective dose” of a drug is an amount of a drug that, when administered to a subject, will have the intended therapeutic effect, for example, alleviation, amelioration, palliation or elimination of one or more manifestations of neurotoxin-induced paralysis or residual neuromuscular blockade residual in the subject. A person of ordinary skill in the art will be able without undue experimentation, having regard to that skill and this disclosure, to determine a therapeutically effective amount of a particular AChl, mAChR antagonist, AChE restoring agent, other agent or combination of agents for practice of this invention.

1.13 Abbreviations: “ACh” is an abbreviation for acetylcholine; “AChl” is an abbreviation for acetylcholinesterase inhibitor; “mAChR” is an abbreviation for muscarinic acetylcholine receptor; “nAChR” is an abbreviation for nicotinic acetylcholine receptor; “RNMB” is an abbreviation for residual neuromuscular blockade; “NNMBA” is an abbreviation for nondepolarizing neuromuscular blocking agent.

### 2. Targets and Agents related to Acetylcholine Signaling

### 2.1 Acetylcholine (ACh)

Acetylcholine (ACh) is a neurotransmitter synthesized in the cytoplasm of nerve cells. When an action potential reaches a nerve ending, a vesicle releases acetylcholine into a synapse. Once in the synapse, acetylcholine diffuses across the synaptic cleft and binds with a post-synaptic acetylcholine receptor. The binding of acetylcholine to its receptor triggers depolarization of the post-synaptic cell. The receptor mediated response is subsequently terminated when acetylcholine is hydrolyzed by an acetylcholinesterase to acetic acid and choline.

### 2.2 Acetylcholinesterase (AchE)

Acetylcholinesterase (EC 3.1.1.7) is a serine protease that hydrolyzes acetylcholine. Assays for acetylcholinesterase activity are known (see, e.g., EIIman et al, Biochem. Pharmacol., 7, 88-95, 1961).

### 2.3 Acetylcholine Receptors (AChR)

Acetylcholine binds to two main types of receptors, the nicotinic acetylcholine receptor (nAChR) and the muscarinic acetylcholine receptor (mAChR). Nicotinic acetylcholine receptors are generally found in the plasma membranes of certain neurons and on the postsynaptic side of neuromuscular junctions (which controls skeletal muscles). Muscarinic acetylcholine receptors are generally found in organs involved in the parasympathetic nervous system.

### 2.4 Acetylcholinesterase Inhibitors (AChls)

The deleterious effects of neurotoxic venoms, or residual effects of nondepolarizing neuromuscular blocking agents (NNBAs), can be counteracted by inhibiting acetylcholinesterase at the neuromuscular junction. Acetylcholinesterase terminates the nAChR response by hydrolyzing ACh to acetic acid and choline. Without intending to be bound by a specific mechanism, inhibiting acetylcholinesterase activity prevents the hydrolysis of ACh, which increases the effective ACh concentration in the neuromuscular junction and thereby ameliorates the effect of the α-neurotoxins and other neurotoxins such as β-neurotoxins, or residual effects of NNBAs.

Acetylcholinesterase activity can be inhibited by administering an acetylcholinesterase inhibitor. The term “anti-acetylcholinesterase” is used interchangeably with “acetylcholinesterase inhibitor” (and does not refer to use of an immunoglobulin). Preferably the AChl is a reversible inhibitor. However, in particular embodiments a quasi-reversible or irreversible inhibitor is used. Preferably the active moiety AChl is a small molecule (MW<1000).

Illustrative examples of reversible acetylcholinesterase inhibitors include: ambenonium; demarcarium; donepezil; edrophonium; galantamine; huperzine A; ladostigil; lactucopicrin; neostigmine; physostigmine; pyridostigmine; rivastigmine; tacrine; phospholine iodide; and ungeremine. Other acetylcholinesterase inhibitors are used, or may be developed in the future. In some embodiments, the AchI has at least about 90%, at least about 100%, or at least about 150% of the inhibitory activity, on a molar basis, as neostigmine in an inhibition assay.

In some embodiments, the anticholinesterase is phospholine iodide, physostigmine or pyridostigmine. Exemplary forms are solutions or suspensions containing inhibitor at a concentration of 0.1 mg/mL to 100 mg/mL or more. In some embodiments, the anticholinesterase is phospholine iodide (echothiophate), physostigmine or pyridostigmine in concentrations ranging from 0.125%-0.25%. Phospholine iodide is an irreversible acetylcholine inhibitor that has previously been used to treat glaucoma, and may be used the treatment of neurotoxic envenomation according to the invention. See, e.g., Deroetth et al., 1965, Effect of Phospholine Iodide on Blood Cholinesterase Levels of Normal and Glaucoma Subjects. Am J Ophthalmol 59: 586-592; De Roetth et al., 1966, Blood cholinesterase activity of glaucoma patients treated with phospholine iodide. Am J Ophthalmol 62: 834-838, Hiscox et al., 1966, The effect of echothiophate iodide on systemic cholinesterase. Can J Ophthalmol 1: 274-282; Axelsson et al., 1970, Side effects from use of long-acting cholinesterase inhibitors in young persons. Acta Ophthalmol (Copenh) 48: 396-400. Other anticholinesterases approved in the US or elsewhere for administration in eye drops for other conditions may be used for treatment of neurotoxic snakebite. Because of the nature of potentially lethal effect of snake bite, even drugs associated with some level of toxicity may be suitable.

In some embodiments the inhibitor is conjugated to another molecule such as a biocompatible and/or biodegradable nanoparticle. In some embodiments, the anticholinesterase is combined with another drug that combats the hemotoxic effects of complex venoms and prevents clotting disorders by preventing the consumption of fibrin or other clotting factors alone or in combinations such as mixtures or conjugates. In some embodiments the antidote is combined with herbal extracts or other compounds that inhibit phospholipase A2 preventing clotting disorders and degradation of the pre-synaptic neurons at the neuromuscular junction. In some embodiments, the combined antidotes are combined with permeation enhancers.

In some embodiments, the acetylcholinesterase inhibitor is irreversible (e.g., echothiophate, an organophosphorous ACE inhibitor, or other irreversible or quasi-irreversible inhibitor or acetylcholinesterase).

2.5 Muscarinic Acetylcholine Receptor Antagonists (mAChR Antagonists)

Administration of an acetylcholinesterase inhibitor prolongs the action of acetylcholine at muscarinic acetylcholine receptors (mAChRs). mAChR antagonists (also known as “anticholinergic” agents) may optionally be administered to the subject to whom acetylcholinesterase inhibitors are administered. mACHR antagonists block muscarinic receptors, thus inhibiting cholinergic transmission. Illustrative examples of mAChR antagonists include: atropine; benzatropine; glycopyrrolate; ipratropium; mebeverine; oxybutynin; pirenzepine; scopolamine; tiotropium; and tropicamide. In some embodiments, the antimuscarinic is glycopyrrolate.

### 2.6 Acetylcholinesterase Reactivating Agents

Acetylcholinesterase reactivating agents (also called acetylcholinesterase restoring agents) are known in the art. See, e.g., Luo et al., 2007, “An in vitro comparative study on the reactivation of nerve agent-inhibited guinea pig and human acetylcholinesterases by oximes,” Biochemistry 23; 46(42):11771-9, incorporated herein by reference. Example of reactivating agents that may be used in the practice of the present invention include oxime derived acetylcholinesterase reactivating agents, such as pralidoxime.

2.7 Nondepolarizing Neuromuscular Blocking Agents Compete with ACh for Binding to nAChRs

Nondepolarizing neuromuscular blocking agents (NNBAs) compete with ACh for binding to nicotinic acetylcholine receptors, and are commonly used in clinical and veterinary anesthesia. Exemplary NNBAs include, for example and not limitation, rapacuronium (Raplon); mivacurium (Mivacron); atracurium (Tracrium); doxacurium (Nuromax); cisatracurium (Nimbex); vecuronium (Norcuron); rocuronium (Zemuron); pancuronium (Pavulon); tubocurarine (Jexin); pallamine (Flaxedil); pipecuronium; and vecronium.

### 3. Administration of Acetylcholinesterase Inhibitors in Response to Snake Bite

Neurotoxins (such as α-neurotoxins) found in snake venom compete with or block ACh for binding to nicotinic acetylcholine receptors. Most deaths from acetylcholine-mediated neurotoxins are caused by skeletal muscle paralysis. This triggers respiratory failure and unless the victim is treated, results in death. In general, the mechanism of action of these neurotoxins is the disruption of the normal function of the nAChR by decreasing the effective concentration of ACh that is available for binding to the neuromuscular junction. This occurs because neurotoxins are antagonists of nAChR and compete with ACh for the nAChR binding site or damage the synapse itself, compromising the ability of the neuron to release ACh. The severity of the physiological response of the venom/neurotoxin is directly correlated with the affinity of the neurotoxin for the nAChR or the nerve terminals responsible for releasing ACh or both.

In one aspect, the invention provides a method for treating or reducing the likelihood of neurotoxin-induced respiratory failure in a human subject by determining that the subject is a victim of a snake bite and administering a pharmaceutically effective dose of an acetylcholinesterase inhibitor to the subject, wherein the administration is not via injection.

In some embodiments the subject treated with an acetylcholinesterase inhibitor is also treated an mAChR antagonist. In some embodiments the subject treated with an acetylcholinesterase inhibitor without any administration of an mAChR antagonist. In some embodiments edrophonium is not administered to the subject prior to administration of neostigmine or other AChl. In some embodiments edrophonium is not administered at any time during the course of treatment with AChl.

The methods may be carried out using any of a variety of acetylcholinesterase inhibitors. When the subject is human, it is preferred that the acetylcholinesterase inhibitor is approved in the U.S. and/or Europe and/or Australia for administration to humans.

In some embodiments a subject is treated with an acetylcholinesterase inhibitor and a mAChR antagonist administered together. The AChl and mAChR antagonist may be administered as an admixture, a solution comprising both agents, and the like. Typically the mixture comprises a pharmaceutically acceptable carrier. Preferably the mixture comprises the AChl and mAChR antagonist at a weight or molar ratio so that administration of a given volume delivers a therapeutically effective dose of each agent. In some embodiments the AChl is selected from ambenonium; demarcarium; donepezil; edrophonium; galantamine; huperzine A; ladostigil; lactucopicrin; neostigmine; physostigmine; pyridostigmine; rivastigmine; tacrine; phospholine iodide; or ungeremine. In some embodiments the mAChR antagonist is selected from atropine; benzatropine; glycopyrrolate; ipratropium; mebeverine; oxybutynin; pirenzepine; scopolamine; tiotropium; and tropicamide.

### 3.1 Subjects to Whom Acetylcholinesterase Inhibitors are Administered to Treat or Reduce the Likelihood of Neurotoxin-Induced Respiratory Failure

In one embodiment of the invention, an acetylcholinesterase inhibitor is administered to a subject who is an identified as a victim of snake bite. In some embodiments administration occurs prior to determination that envenomation occurred, or prior to in determination that neurotoxic envenomation has occurred. Thus, in some embodiments an acetylcholinesterase inhibitor is administered to a subject who is a identified as a victim of snake bite in which envenomation occurred. In some embodiments an acetylcholinesterase inhibitor is administered to a subject who is a identified as a victim of envenomation by snake with a neurotoxic venom.

There are a number of ways to determine that a subject is a victim of snake bite. These include:

(a) the subject or another person witnessed the bite;

(b) physical evidence of snake bite (e.g., puncture wounds or lacerations, localized pain, local redness or swelling) is observed;

(c) the subject exhibits signs or symptoms consistent with snake bite envenomation (e.g., pain, redness, bleeding, or other evidence of envenomation);

(d) the subject exhibits signs or symptoms consistent with neurotoxic envenomation and has not been previously diagnosed with a condition other than neurotoxic envenomation that accounts for the signs or symptoms.

(e) venom has been detected (e.g., at the bite site, in urine or blood, using a snakebite venom detection kit).

In one embodiment, the step of determining that the subject is a victim of a snake bite comprises determining the subject is a victim of a bite from a neurotoxic venomous snake bite. This can be done by, for example, visual identification of the snake or identification of the snake using physical indicia identifying the type of snake. In some cases it will be possible to deduce that the subject is a victim a bite from a neurotoxic venomous snake when the snake bite occurs in a locale in which the venomous snakes are very commonly found and non-venomous snakes are relatively rare or where there are signs or symptoms consistent neurotoxic envenomation.

In some cases the subject exhibits signs or symptoms of neurotoxic envenomation. Signs and symptoms (i.e., clinical effects) of neurotoxic envenomation include paresthesia, drowsiness, dysconjugate gaze, small muscle paralysis which may result in ptosis (lid lag), weakness of neck muscles, dysphagia, mydriasis, fasiciculation, increased salivation, increased sweating, loss of muscle coordination, abdominal pain, difficulty speaking, nausea, difficulty swallowing and other bulbar palsies, and vomiting, hypotension, respiratory distress and respiratory muscle paralyses. In some cases the subject displays early signs of including early signs of neurotoxic envenomation, such as small muscle paralysis in the form of lid lag, dysconjugate gaze, difficulty swallowing and other bulbar palsies.

In some cases the acetylcholinesterase inhibitor is administered to a subject who does not exhibit symptoms of neurotoxic envenomation, such as a subject for whom there is evidence or a snake bite, but for whom there is insufficient evidence to exclude neurotoxic envenomation.

An acetylcholinesterase inhibitor is not administered in cases in which there is evidence of snake bite but in which neurotoxic envenomation can be excluded. For example, an acetylcholinesterase inhibitor is generally not administered where it is clear, based on symptoms, sighting of a snake, or location, that the snake bite is from a non-venomous snake or from a snake that delivers a non-neurotoxic venom.

Administration of an acetylcholinesterase inhibitor may provide little benefit to subject who is victim of a snake bite, but not envenomation (e.g., a “dry” snake bite) or of victim of a envenomation with a venom that is not non-neurotoxic (e.g., hemotoxic venom). However, under the conditions in which the invention is used it may not be possible to determine whether envenomation or neurotoxic envenomation has occurred prior to the onset of paralysis. In general it may be detrimental to the subject to delay treatment while a determination is made.

It will be recognized that when viewed prospectively, the likelihood of neurotoxin-induced respiratory failure in the subject is reduced even when a snake bite victim is ultimately determined not to have neurotoxic envenomation and it becomes apparent retrospectively that the administration did not prevent or reverse the onset of paralysis.

3.2 Other Therapeutic Agents

In some embodiments the patient receiving intra-nasal and ocular administration of an acetylcholinesterase inhibitor is treated with other agents, such as anti-venom, a mAChR antagonist, an acetylcholinesterase reactivating agent, and other agents (e.g., phospholipase inhibitors).

Treatment with anti-venom may be particularly appropriate in the case of envenomations that have (or may have) both neurotoxic and hemotoxic components, causing both paralysis and bleeding or clotting disorders. In various embodiments, acetylcholinesterase inhibitor is administered concurrently with, prior to, or following administration of antivenom. In some embodiments, acetylcholinesterase inhibitor is administered prior to administration of antivenom (e.g., more than 1 hour prior to administration of antivenom) such as, for example, when acetylcholinesterase inhibitor is administered prior to the time antivenom is available. In some embodiments, acetylcholinesterase inhibitor is administered after administration of antivenom (e.g., more than 1 hour after first administration of anti-venom, or following completion of a course of treatment of anti-venom) for example, when anti-venom treatment does not result in resolution of symptoms.

In some embodiments, the patient treated with acetylcholinesterase inhibitor is not treated with antivenom. For example, the patient treated with acetylcholinesterase inhibitor is not treated with antivenom in the 24-hour period, alternatively the 48-hour or 96-hour period prior to administration with acetylcholinesterase inhibitor. In some embodiments the patient treated with acetylcholinesterase inhibitor is not treated with antivenom during the course of treatment. In some embodiments, acetylcholinesterase inhibitor is in a course of therapy that includes antivenom.

In some embodiments, acetylcholinesterase inhibitor is administered according to the invention in combination with (i.e., in the same course of therapy with inhibitors of other venom enzymes, such as inhibitors of phospholipases such as phospholipase A2, other and other enzymes that can cause paralysis, destroy nerve terminals and/or cause bleeding disorders are inhibited to prevent or delay death. In some aspects the invention is combined with inhibitors of the enzymes stimulated by other components of venom, such as melittin, which stimulates phospholipase and can be both hemotoxic and neurotoxic (Clapp et al, 1995, Brain Res. 693:101-11).

In some aspects, one or more components of the invention are combined or conjugated with antivenom or fragments of antibodies directed against venom components. In any of these combinations might be added mACHR antagonists such as atropine, glycopyrrolate or others and permeability enhancing agents alone or in combination.

3.3. Administration in Response to Venomous Bites and Stings Other than Snake Bite

An acetylcholinesterase inhibitor and optionally a mAChR antagonist also may be administered, as described above to treat or reduce the likelihood of neurotoxin-induced respiratory failure following envenomation by venomous arthropods such as Centuroides spp stings (wood scorpion), cone snails and tropical jellyfish.

### 4. Intra-Nasal and Ocular Administration to Treat or Prevent Residual Neuromuscular Blockade (RNMB) and Shorten Time of Assisted Respiration Via Mask or Endotracheal Intubation

In one aspect of the invention, an acetylcholinesterase inhibitor is administered to a patient to whom a nondepolarizing neuromuscular blocking agent has been administered, such as a surgical patient, to treat or reduce the likelihood of incomplete neuromuscular recovery. Typically the acetylcholinesterase inhibitor is administered intra-nasally, or ocularly. The inhibitor may be administered by mask.

Nondepolarizing neuromuscular blocking agents (NNBAs) are used during surgery and other procedures to provide muscular relaxation and reduce coughing, gagging and blinking. Although NNBAs provide significant benefit, there are also associated with undesirable post-operative complications. See Murphy et al., Anesthesia & Analgesia July 2010 Vol. 111 No. 1 pp. 120-128; Kopman, 2008, Anesthesiology 109:363-64, and Plaud et al, 2010, Anesthesiology 112: 1013-1022, each incorporated herein by reference. These complications may arise from incomplete metabolism of NNBAs. Incomplete neuromuscular recovery during the early postoperative period may result in acute respiratory events (hypoxemia and airway obstruction), unpleasant symptoms of muscle weakness, longer post anesthesia care unit stays, delays in tracheal extubation.

According to the present invention, an acetylcholinesterase inhibitor (AChl) is administered to the patient following completion of surgery or other procedure for which an NNBA was administered (e.g., endotracheal intubation). In some embodiments, the AChl is administered when it is necessary to reverse a neuromuscular blockade to affect recovery or facilitate neurological testing. The AChl can be administered intra-nasally or ocularly as described supra. In one embodiment, AChl (e.g., aerosolized neostigmine) can be administered intra-nasally by mask or in line with standard oxygen tubing nebulization chamber and aerosol mask. AChl can be administered continuously or in discrete doses. In some cases, the AChl is administered intermittently over short periods as 1 to 10 minutes, or continuously for 1 to 30 minutes, with or without supplemental oxygen, steroids, epinephrine or mAChR antagonists such as atropine. Exemplary guidelines for administration (dose, formulation, frequency, etc.) are provided supra in §2 and is applicable to the administration of AChls to treat or prevent residual neuromuscular blockade. However, those of ordinary skill in the art can use routine methods to optimize dose and administration.

In one aspect of the invention, acetylcholinesterase inhibitor is administered to a patient post-operatively, e.g., via nasal or ocular routes, for a period of at least 12 hours, at least 24 hours, or at least 36 hours post-operatively, at, for illustration, a frequency of such as about once every 0.25 hours, 0.5 hours, 1 hour, 2 hours, 4 hours, 6 hours.

This method may be used routinely as a highly effective means of insuring against undetected residual paralysis. The AChl also may be administered, according to the invention, on a routine basis to reverse paralysis in the recovery room without the need for a balancing agent such as an mAChR antagonist such as atropine or glycopyrrolate.

Plaud et al., supra, describes measurement of and definitions of residual paralysis. Plaud suggests that adequate neuromuscular recovery (i.e., the absence of residual paralysis) may be characterized by a train-of-four stimulation ratio (TOFR), and that a TOFR more than or equal to 1.0 is considered adequate. According to the present invention the AChl can be administered intra-nasally or ocularly to a patient with no apparent paralysis, such as a patient with a TOFR equal to or greater than 1.0 or with an unknown (unmeasured) TOFR.

Intra-nasal or ocular administration of acetylcholinesterase inhibitors also may be used when patients are extubated prematurely, for example or during awake neuroanesthesia. Likewise, intra-nasal or ocular administration of acetylcholinesterase inhibitors also may be used if there is a malfunction of the IV and it would be difficult to titrate a reversal agent with an anticholinergic agent. This would aid patients' recovery times and help them regain their breathing ability faster without the dangerous cardiovascular effects of intra venous formulations. The therapeutic safety window would be greater reducing the chances of medical error and direct toxicity of the anticholinesterases to the heart, gut and mucous membranes.

In a related embodiment acetylcholinesterase inhibitors are administered to relieve deep blockade (e.g., TOF less than 0.2 or equivalent).

In some embodiments, both acetylcholinesterase inhibitors and mAChR agonists are administered.

### 5. Type, Dosage, Frequency, and Formulations for Administration of AChls Type

For administration of an acetylcholinesterase inhibitors according to the invention, any of the AChls described herein may be used, as well as AChls developed or discovered in the future may be used. In some embodiments the acetylcholinesterase inhibitor is a drug that has been approved by the Federal Drug Administration or an equivalent regulatory body.

In some embodiments, the acetylcholinesterase inhibitor is a reversible inhibitor (for example, neostigmine, physostigmine, or pyridostigmine). In some embodiments, the acetylcholinesterase inhibitor used is a quasi-reversible or irreversible antagonist of the enzyme acetylcholinesterase. It is preferred that the acetylcholinesterase inhibitor is readily bioavailable. In one embodiment the acetylcholinesterase inhibitor is neostigmine. In another embodiment the acetylcholinesterase inhibitor is physostigmine. In another embodiment the acetylcholinesterase inhibitor is pyridostigmine.

Dose

The dose of acetylcholinesterase inhibitors administered will depend on the particular inhibitor used, the form in which it is administered (e.g., powder, spray, or aerosol), formulation (e.g., the presence or absence of a permeability enhancer), and other factors know to those of ordinary skill in the pharmacology and the route of administration (e.g., ocular or nasal). In general, intranasal dose is from 1 microgram to 100 mg per dose, generally in the range of 0.1 mg to 200 mg, often in the range of about 1 to 100 mg per dose. In general, the intranasal dose for a particular agent is higher than the standard intravenous dose (e.g., by 2-fold to 10-fold or more). It will be appreciated that doses will vary depending on factors such as the subject's age, size, gender and response to treatment.

Intranasal administration of neostigmine in myasthenic patients was described by Sghirlanzoni et al., 1992, J Neurol. 239:165-9 and Ricciardi et al., 1991, J. Neurol Neurosurg Psychiatry. 54:1061-2 using 6% neostigmine methylsulfate in individual doses spaced by 15 minutes and in alternating nostrils. Patients saw salutary effects usually after the first dose, but requiring and tolerating without ill effect up to 5 puffs of intranasal (IN) neostigmine at 15 minute intervals. In one embodiment, a 5% neostigmine solution is used.

In some embodiments the anticholinesterase, alone or in combination with an anticholinergic agent such as atropine, is administered to the eye (“ocular administration”). In one approach the drug is administered as an eye drop.

Eye drops may be administered with or without an anticholinergic agent such as atropine. Administration would occur at the time of the bite, just after the bite or at the onset of symptoms, or as adjunctive treatment with antivenom and other supportive treatments in the pre-hospital or hospital setting. In one embodiment, 1 to 10 drops of solution containing the anticholinesterase are be instilled in the medial canthus of each eye with the eyelids retracted allowing maximum absorption of the drug.

The drug may be formulated with a carrier such as DMSO, citric acid, sodium citrate, benzalkonium chloride, liposomes or other delivery vehicles. The drug may be conjugated in such a manner that it could be administered in lower doses with higher specificity of targeting affected parts of the nervous system. Drops would generally be administered in 1-hour intervals as needed until initial effects were seen and then once the patient showed signs of recovery maintained by dosing every 4 hours as with the nasal spray or aerosolized anticholinesterase/anticholinesterase-anticholinergic mixture.

Higher doses may be given in a veterinary context, for example, when an AChl is administered to a large animal.

Frequency

Generally the acetylcholinesterase inhibitor is administered as soon as possible following identification of the subject. If the subject exhibits signs of neurotoxin envenomation the acetylcholinesterase inhibitor is preferably administered immediately. If no signs have appeared administration may optionally be delayed until the earliest signs of paralysis (e.g., lid lag) are observed or there is reason to believe it is a venomous bite for other reasons, such as pain, shortness of breath, bleeding, bruising or the snake is identified as being a one known to inject neurotoxins.

In some cases, the acetylcholinesterase inhibitor is administered one time for snake bite. However, multiple administrations may be indicated over time, depending on the patient's response (e.g. appearance or progression of signs of paralysis). For example, a 1%-10% neostigmine solution (e.g., 1%-6%, or about 5%) may be administered at 15 minute intervals for an hour or longer. Administration up to 6 or more times per day is contemplated (e.g., once every four). It will be appreciated that a patient treated in a clinic or hospital can self-administer inhibitor after discharge. In one aspect of the invention, an acetylcholinesterase inhibitor, or mixture of AChl and anticholinergic agent is administered, e.g., via nasal or ocular routes, for a period of at least 12 hours, at least 24 hours, or at least 36 hours post-operatively, at, for illustration, a frequency of such as about once every 0.25 hours, 0.5 hours, 1 hour, 2 hours, 4 hours, 6 hours.

Formulation

Formulations suitable for intra-nasal administration of drugs are known, and it is within the ability of one of ordinary skill in the art to formulate AChls for nasal administration. In some embodiments, the drug is formulated with a mucosal adsorption enhancer such as DMSO, citric acid, sodium citrate, propylene glycol, glycerin, L-ascorbic acid, sodium metabisulfite, edetate disodium, benzalkonium chloride, sodium hydroxide, dimethylformamide, ethanol, propylene glycol, 1,3 butanediol, 2-pyrrolidones and mixtures thereof. Other mucosal adsorption enhancers are known in the art, including those described in US Pat. Publication 2007/0026079 to Herlands et al., incorporated herein by reference. Also see Constantino et al., 2008, BMC Neuroscience 9(Suppl 3):S6, incorporated herein by reference.

Information about intra-nasal formulations, enhancers, dose, frequency, and examples of acetylcholinesterase inhibitors administered by an intra-nasal route is found in Quay et al., US Patent Publication No. 2006/0003989 “Compositions and methods using acetylcholinesterase (ACE) inhibitors to treat central nervous system (CNS) disorders in mammals,” which is incorporated herein by reference in its entirety. Also see Sghirlanzoni et al., 1992, “Efficacy of intranasal administration of neostigmine in myasthenic patients.” J Neurol. 239:165-9, incorporated herein by reference.

Formulations for ocular administration are well known in the art. Exemplary are saline and phosphate buffered saline, optionally with a preservative.

### 6. Delivery Systems

Any suitable method of intranasal delivery can be employed for delivery of acetylcholinesterase inhibitors (or other compounds). The drug may be administered as a solution, as a powder, encapsulated in liposomes or conjugated with other molecules and the like. The drug can be administered as nasal drops, nasal sprays, nasal powders, aerosols, nasal gel, or any other intra-nasal dosage form.

In some embodiments, the intra-nasal drug delivery device is an inhaler or nebulizer device. In some embodiments the device is an MDI, a hybrid MDIs/nasal spray or droppers. In some embodiments, the intra-nasal drug delivery device is an intra-nasal mucosal atomization device. Atomization prepares medication in soluble particles that are optimal size for absorption through the nasal mucosa (2-10 micrometers). See Mygind et al., Rhinology 1978; 16(2): 79-88, incorporated herein by reference. Several commercially available devices exist today for atomization of medication for IN delivery, including the Accuspray Nasal Atomizer™, the MAD (Mucosal Atomization Device™), the Optinose™, and the ViaNasa Electronic Atomizer™. In other embodiments, the intra-nasal drug delivery device is a dropper. In other embodiments, the intra-nasal delivery device is a metered nasal sprayer. In other embodiments intra-nasal administration is carried out using a tampon, sponge, insufflator or pump. Information about pressurized devices used for aerosol inhalation drug delivery is also provided in Remington: The Science and Practice of Pharmacy, 19^{th} Ed., incorporated herein by reference, at Chapter 95 “Aerosols”, and Chapter 41, “Drug Absorption, Action and Disposition.”

A device that administers a metered dose may be used. In some embodiments the device delivers a single unit dose of the drug or drugs. In some embodiments the device is disposable. In some embodiments the device is refillable.

Optionally, the delivery system may be a disposable device capable of providing a single metered dose or from 1 to 5 metered doses.

An AChl can be intra-nasally administered in aerosolized form in line with standard oxygen tubing nebulization chamber and aerosol mask. AChl can be administered continuously or in discrete doses. In some cases, the AChl is administered intermittently over short periods as 1 to 10 minutes or continuously for 1 to 30 minutes with or without supplemental oxygen, steroids, epinephrine or mAChR antagonists such as atropine that can be administered in aerosol.

Any suitable method of ocular delivery can be employed for delivery of acetylcholinesterase inhibitors (or other compounds). Typically a dropper is used to administer solution to the eye.

### 7. Administration of mAChR Antagonists

Acetylcholinesterase inhibitors prolong the action of ACh at the muscarinic acetylcholine receptors (mAChRs) as well as nicotinic acetylcholine receptors (nAChRs). mAChRs are generally found in organs in the parasympathetic nervous system. When the effective concentration of ACh is increased with respect to mAChRs, it results in a passive discharge of the parasympathetic nervous system. If the magnitude of this discharge is sufficient to trigger a large, the subject may experience one or more of a collection of symptoms often referred to as “SLUDGE.” SLUDGE refers to: (i) salivation from the stimulation of the salivary glands; (ii) lacrimation from the stimulation of the lacrimal glands; (iii) urination from the relaxation of the internal sphincter muscle of urethra and contraction of the detrusor muscles; (iv) defecation from the relaxation of the internal anal sphincter; (v) gastrointestinal upset including diarrhea from changes in the smooth muscle of the GI tract; and (vi) emesis.

To prevent this massive discharge when acetylcholinesterase inhibitors are administered intravenously, a competitive antagonist of mAChR also may be administered to mitigate the physiological responses of the parasympathetic nervous system.

In certain embodiments of the present invention ACh agonists are administered to the snake bite victim receiving AChls. Thus in one aspect of the present invention, a method of treating a neurotoxin-induced respiratory failure is provided which comprises: identifying a victim who has been delivered a dose of venom by an animal and is suffering from clinical effects of envenomation, including early signs such as small muscle paralysis to deadly ones such as respiratory failure; administering a pharmaceutically effective dose to the victim of an acetylcholinesterase inhibitor (e.g., intra-nasally); and administering (e.g., intra-nasally) a pharmaceutically effective dose to the victim of a mAChR antagonist. In a related aspect, the invention provides a method for treating or reducing the likelihood of neurotoxin-induced respiratory failure in a human or animal subject by determining that the subject is a victim of a snake bite; administering (e.g., intra-nasally) a pharmaceutically effective dose of an acetylcholinesterase inhibitor to the subject; and administering (e.g., intra-nasally) a pharmaceutically effective dose of an muscarinic acetylcholine receptor agonist to the subject.

For intra-nasal or ocular administration of an mAChR agonists, without limitation, any of the agonists in §2.5, supro may be used. When used in humans, it is preferred that mAChR agonist is a drug that has been approved by the Federal Drug Administration or an equivalent regulatory body.

In some embodiments, the mAChR antagonist used is according to the invention is a competitive antagonist. In other embodiments, the mAChR antagonist is a reversible competitive antagonist. In preferred embodiments, the mAChR antagonist does not cross the blood brain barrier. In preferred embodiments, the mAChR antagonist is selective for mAChR over the nAChR. Preferably, the mAChR competitive antagonist is also short acting (e.g., having a half-life of 4 to 6 hours or less). In some embodiments, the mAChR agonist is glycopyrrolate or atropine. In these embodiments, the acetylcholinesterase inhibitor is neostigmine and the mAChR antagonist is glycopyrrolate or atropine.

In various embodiments, the ACh inhibitor and the mAChR antagonist are administered at about the same time in either order (e.g., within 10 minutes, preferably within 5 minutes, of each other or simultaneously in a premade mixture). In some embodiments, the ACh inhibitor is administered first, and the mAChR agonist administered after a lag of about 5 minutes or as needed to reverse undesired muscarinic anticholinergic effects such as gastrointestinal upset or hypersalivation.

In general, intranasal dose of the mAChR antagonist is from 100 micrograms to 10 grams per dose, generally in the range of 0.1 mg to 100 mg, often in the range of about 1 to 50 mg per dose, and often in the range of 1.5 to 12 mg per dose.

In some embodiments the ACh inhibitor and mAChR antagonist are administered simultaneously (e.g., inhaled simultaneously from two compartments of a single deliver device) or as a mixture. Several studies have been done showing intravenous administration of mixtures of ACh inhibitor and mAChR agonist. See, e.g., Mirakhur et al. Reversal of neuromuscular blockade: dose determination studies with atropine and glycopyrrolate given before or in a mixture with neostigmine. Anesth Analg. 1981 August;60(8):557-62. It is within the ability of those of ordinary skill in the art, guided by the medical and pharmacological literature, to optimize dosing and dosing intervals. mAChR antagonists can be administered using the methods and devices described supra in §2 for administration of ACh inhibitors.

### 8. Intra-Nasal Administration of Acetylcholinesterase Inhibitors in the Absence of Administration of mAChR Antagonists

In one aspect, the invention provides a method for treating or reducing the likelihood of neurotoxin-induced respiratory failure in a human subject by determining that the subject is a victim of a snake bite, envenomation, or neurotoxic envenomation, intra-nasally administering a pharmaceutically effective dose of an acetylcholinesterase inhibitor, and not administering a mAChR agonist to the subject. This is contrary to the usual approach in the art. For example, WHO guidelines for the treatment of snakebite by intravenous administration of anticholinesterase inhibitor indicate the that atropine, glycopyrrolate or other mAChR antagonist should be administered. Likewise, Wilson et al., supra, notes “in order to minimize these parasympathetic effects, anticholinergic medications, including atropine and glycopyrrolate, must be administered along with the neostigmine.” Page e3, emphasis added.

### 9. Administration of AChE Reactivating Agent

In some embodiments, the ACh inhibitor is irreversible or quasi-irreversible (e.g. phospholine iodide) and is administered with an oxime-derived AChE restoring agent such as pralidoxime most likely with, but possibly without an mAChR inhibitor such as atropine or biperiden.

### 10. Devices, Kits and Dosage Forms

In another aspect of the present invention, a kit is provided which comprises an acetycholinesterase inhibitor, a drug delivery device, and instructions for administration in response to envenomation. In some embodiments, the delivery device is adapted for intra-nasal administration (intranasal drug delivery device). In some embodiments, the delivery device is adapted for ocular administration (ocular drug delivery device).

In some embodiments, a kit is provided which comprises an acetycholinesterase inhibitor, a mAChR antagonist, and a drug delivery device. In some embodiments, a kit is provided which comprises an acetycholinesterase inhibitor, a mAChR antagonist and/or a AChl restoring agent mAChR antagonist, and a drug delivery device In some embodiments the acetycholinesterase inhibitor, mAChR antagonist and/or AChl restoring agent are in separate devices in the same kit. In some embodiments the acetycholinesterase inhibitor and mAChR antagonist are provided as a mixture. In some embodiments the acetycholinesterase inhibitor and AChl restoring agent are provided as a mixture.

In some embodiments, the intranasal drug delivery device is an intranasal mucosal atomization device or nebulizer. In some embodiments, the intranasal drug delivery device delivers an aerosol. In some embodiments, the intranasal drug delivery device is a dropper for delivering a solution or suspension. In other embodiments, the intranasal delivery device is a is a spray pump device. In some embodiments, the intranasal drug delivery device delivers a metered dose. In some embodiments, the intranasal drug delivery device comprises a pump. In some embodiments, the intranasal drug delivery device is an inhaler. Delivery devices are known in the art and available from commercial suppliers (e.g., Pfeiffer, Germany; Valois, France, Becton Dickinson, France, Nemo, Spain).

In some embodiments, the ocular drug delivery device is a dropper. In some embodiments, the ocular drug delivery device delivers a metered dose.

In some embodiments, drug(s) is provided as a solution, suspension, gel, powder or other form. Individual drugs or a mixture of drugs (e.g., AChl and mAChR antagonist) can be compounded into a medicament in accordance with generally accepted procedures for the preparation of pharmaceutical preparations, as described in standard textbooks on the subject See, for example, Pharmaceutical Preformulation and Formulation A Practical Guide from Candidate Drug Selection to Commercial Dosage Form, M Gibson ed., Informa Health Care 2009, Pharmaceutical Manufacturing Handbook Production and Processes, S C Gad ed., Wiley-lnterscience 2008, and the latest edition of Remington's Pharmaceutical Sciences, Maack Publishing Co, Easton Pa. Steps in the compounding or formulating of the medicament depend in part on the mode of topical administration. Excipients, carriers, buffers and the like are well known in the art. For many applications it is preferable that the drug or mixture is heat stable.

In some embodiments, the drug(s) are prepackaged in the delivery device. In some embodiments, the acetylcholinesterase inhibitor and/or the mAChR antagonist are in dehydrated form and are reconstituted prior to use in the drug delivery device.

In some embodiments, the acetycholinesterase inhibitor and mAChR antagonist are prepackaged in the delivery device. In some embodiments, the device is a single use (disposable) device. In some embodiments, the single use device contains a single dose of acetylcholinesterase inhibitor in a disposable device.

In some embodiments, the drug delivery device contains an inhibitor selected from ambenonium; demarcarium; donepezil; edrophonium; galantamine; huperzine A; ladostigil; lactucopicrin; neostigmine; physostigmine; pyridostigmine; rivastigmine; tacrine; phospholine iodide; and ungeremine. In some embodiments, the drug delivery device contains an agonist selected from atropine; benzatropine; glycopyrrolate; ipratropium; mebeverine; oxybutynin; pirenzepine; scopolamine; tiotropium; and tropicamide.

For illustration and not limitation, examples of combinations include neostigmine 1% to 10%+atropine 0.5 mg to 10 mg; neostigmine 1%-10%+glycopyrrolate 1 mg to 10 mg; neostigmine 1%-10%+biperiden 0.5 mg to 100 mg; pyridostigmine 1 mg to 100 mg+atropine 0.5 mg to 10 mg; pyridostigmine 1 mg to 100 mg+glycopyrrolate 1 mg to 10 mg; pyridostigmine 1 mg to 100 mg+biperiden 0.5 mg to 100 mg.

Also provided is a drug delivery device that comprises an acetycholinesterase inhibitor and optionally a mAChR antagonist, as described herein.

### 11. Other Applications

Methods of the present invention, comprising intra-nasal or ocular administration of acetylcholinesterase inhibitors, and optionally, mAChR antagonists has other applications in human and veterinary health, including national defense.

11.1 Nasal or ocular administration of acetylcholinesterase inhibitors (e.g., neostigmine, physostigmine, or phospholine iodide) also finds use in treating intentional and unintentional anticholinergic overdoses such as diphenhydramine overdose, lomotil overdose, or atropine dosing errors and other overdoses with medications acting on the mAChR and nAChR receptors to cause neurotoxic effects. Advantageously, using the delivery devices described herein above, administration could begin on site or in ambulances en route to hospital.

11.2 Nasal, ocular or aerosol administration of acetylcholinesterase inhibitors (e.g. neostigmine, distigmine, phospholine iodide also finds use in the context of national defense. In an environment containing weaponized toxins such as botulinum toxin or weaponized mAChR antagonist, soldiers and others such as first responders and emergency care providers can breathe using respirators or gas masks that released at effective doses of the inhibitor, which is thereby administered to the upper respiratory tract which includes intranasal and oropharyngeal delivery and could include, additionally, ocular administration. The inhibitor may be released continuously, periodically, or on command of the individual wearing the gas mask or respirator, by for example, incorporating an atomizer into the device. The dose of inhibitor may be an amount delivered to the oropharyngeal, nasal or ocular mucosal surfaces per hour that is equal to, less than or greater than the parenteral dose per hour such as 5% to 99% or, more often, when given in isolated doses, 2 to 100 times the parenteral doses. Additionally, in combat and during emergency evacuation or air ambulance transport, patients are endotracheally intubated using NMBAs and in the event of an overdose or need to reverse paralysis nasal, aerosol or ocular administration of an AChl could be lifesaving.

11.3 Nasal or ocular administration of acetylcholinesterase inhibitors (e.g., neostigmine or phospholine iodide) also finds use in the treatment of acute urinary retention, bowel evacuation and other dysfunctions mediated by the parasympathetic nervous system (e.g. salivation, lacrimation, defecation, urination, in dentistry for example in the prevention of dental decay by tobacco products and others). See Wang et al., 2009, “Acetylcholinesterase inhibitor is a potentially useful therapeutic agent for nicotine-induced periodontal disease. Med Hypotheses. 73(4):604-5, incorporated by reference herein.

11.4 Nasal or ocular administration of acetylcholinesterase inhibitors (e.g., neostigmine or phospholine iodide) also finds use when it is necessary to restrain patients with anticholinergic mediated-mediated altered mental status or delirium (e.g. diphenhydramine overdose or jimson weed poisoning and the like). By using an ocular or nasal delivery device the use of needles (and risk of needle sticks) can be avoided, making it safer for the patients and the caregivers.

11.5 Nasal or ocular administration of acetylcholinesterase inhibitors (e.g., neostigmine or phospholine iodide) also finds use when it for reversing drug induced bladder obstruction resulting from therapeutic administration of anticholinergic agents (e.g., opioid analgesics) and antihistamines (e.g., diphenhydramine). This will reduce catheter time in bladder or need for urinary catheters, thereby reducing infections, hospitalizations, discomfort and unnecessary trauma to the urethra.

11.6 Nasal or ocular administration of acetylcholinesterase inhibitors (e.g., neostigmine or phospholine iodide) also finds use when it is used to relieve urinary retention and or constipation in paraplegics/quadriplegics and others with neurogenic dysfunction causing inability to void, evacuate stool because of sensory deficits and nerve dysfunction.

### 12. Examples

### 12.1 Example 1

### Response to Snakebite

A subject with known or suspected snakebite exhibits the first signs of weakness in form of lid-lag or other bulbar palsy. A companion, a medical practitioner, or the patient administers or self-administers the intranasal acetylcholinesterase inhibitor and observes for clinical improvement in the form of improved muscle function. Improved muscle function can be determined by qualitatively by subjective improvement in strength, mobility and ease of breathing or by quantitative means such as by electro-myographic techniques and other standardized measures of strength. If the patient's condition deteriorates, then additional doses are given, usually spaced by 15 minutes until the patient either recovers or more common resuscitative techniques are available or needed. Advantageously, and in contrast to conventional methods, the drug may be administered by someone with no or minimal medical training. Advantageously, and in contrast to conventional methods, a subject with known or suspected snakebite can self-administer, even when alone.

### 11.2 Example 2

### Example 2 Intra-Nasal Administration of Glycopyrrolate and Neostigmine

In this experiment, the effect of intra-nasal administration of an mAChR inhibitor (glycopyrrolate) was determined using a healthy male volunteer. 5 cc of glycopyrrolate (0.2 mg/mL) in sterile water was instilled in one nostril using an LMA sponge atomizer. It had no effect on heart rate (range 65 to 72). 5 cc of 0.2 mg/mL glycopyrrolate mixed in DMSO was instilled in the other nostril. There was no notable effect on heart rate. Following administration of the 2nd dose of glycopyrrolate, 3 cc of 1 mg/mL neostigmine was administered into one nostril with no effect on heart rate, no increase in salivation, or any other notable effect. The significance of this is that these medication are well-tolerated and did not change vital signs in a significant manner, consistent with the use of anticholinesterase inhibitor or a combination of anticholinesterase inhibitor and mAChR inhibitor for the purposes above. None of the nasally administered compounds were found to be irritating or cause any discomfort. Glycopyrrolate caused mild drying of the nasal membranes noted prior to administration of neostigmine.

### 11.3 Example 3

### Reversal of Experimental Paralysis in a Human by Intranasal Neostigmine Aerosol

Outline

Intravenous mivacurium is administered to a subject at concentrations of 5-200 mcg/kg/min to induce a safe, stable, low level neuromuscular block for a medical procedure. After completion of the procedure a total of 4 to 30 mg of neostigmine in divided doses—each dose separated by 15 minutes (1 mg/mL of a 5% or a 6% solution) is administered intra-nasally and the regression of the block is followed quantitatively using acceleromyography or clinical measures such as the improvement in muscle strength as measured by thumb adduction, handgrip strength, teeth clenching, head raising and/or swallowing. Reversal or reduction of neuromuscular blockade is evident within 15 minutes of administration of an effective dose of neostigmine.

Establishment and Recording of Neuromuscular Block and Drug Administration

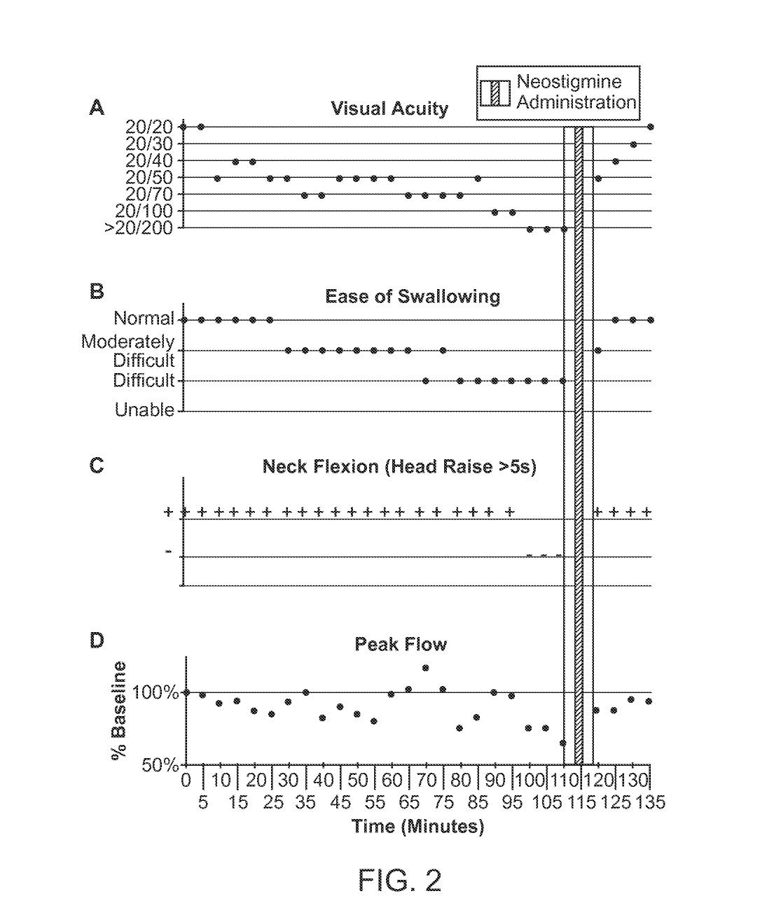

Mivacurium [Mivacron, Oslo, Norway], a curare-like nondepolarizing agent was chosen for the study because earlier studies conducted for other purposes suggested a clinical course that could simulate neurotoxic envenomation. Importantly stable, near steady-state blood concentrations can be reached rapidly compared to other drugs in its class, and its safety profile is good due to its rapid elimination and neuromuscular blockade was achieved by continuous infusion rather than bolus injection as is typical of envenomation[42-44]. Neuromuscular block was quantified by using the train-of-four (TOF) ratio at the left adductor pollicis (AP) muscle measured by acceleromyography and as described previously [43, 44]. When the subject had significant oropharyngeal weakness and met electrophysiological criteria for Level 3 block [43, 44] a single dose of 0.2 g mg IV glycopyrrolate was administered to prevent bradycardia. Five minutes later 6% neostigmine dissolved in sterile water [33] was administered using a primed atomizer (LMA MAD Nasal Device, LMA Corporation North America, San Diego, Calif.). A total dose of 27.6 mg, 0.37 mg/kg with half the volume insufflated in each nostril was given [30, 31, 33, 45] and the subject was left undisturbed for a total period of 10 min (Shaded area, FIG. 2) except for acceleromyographic recording. After the final set of measurements, the mivacurium infusion was terminated and neuromuscular function was allowed to return spontaneously. Emergency equipment and drugs including IV neostigmine and edrophonium (for reversal of mivacurium block) and glycopyrrolate and atropine (for early treatment of neostigmine toxicity) were at the bedside at all times.

Clinical Measures of Muscle Function

Clinical measures of muscle function emphasized those that would be seen in the setting of neurotoxic envenomation that could be readily measured in out-of-hospital settings. The clinical assessments of muscle function were as follows: visual acuity, ease of swallowing [43, 44], ability to protrude the tongue [43, 44], diction [43, 44], and ability to raise the head completely off the bed for more than 5 sec (neck flexion) [43, 44] with a postal scale (WeighMax, Industrial City, Calif., USA) placed under the subjects head to confirm complete elevation and peak respiratory flow measured using a Tru-Zone Peak Flow Meter (Monaghan, Plattsburg, N.Y.). All clinical data were recorded every 5 min throughout the experiment and recorded separately by two physicians who did not communicate with each other or with the anesthesiologist managing the mivacurium infusion and acceleromyographic TOF ratio recordings. The subject was blinded to all clinical and acceleromyographic data as well as to the mivacurium infusion rate and the levels of neuromuscular blockade.

Data Analysis

The mivacurium infusion was maintained with unchanged infusion rate, enabling the investigators to determine the characteristics of neostigmine effect [43]. Analysis of TOF ratios were made using TOFMON software (Schering-Plough Corporation, Kenilworth, N.J.) and considered stable if the values obtained 10 min apart differed less than 5% and accounting for repeated measures [43, 44]. Student's t-test results were calculated and reported as average values, standard deviation (SD), and 95% confidence intervals (95% CI) [43, 44, 46].

Results

During administration of the mivacurium, the subject experienced progressive weakness mimicking paralysis from neurotoxic envenomation, including loss of visual acuity, difficulty swallowing, jaw ptosis, tongue weakness, inability to flex the neck, and the beginnings of breathing difficulty. The subject was always fully awake and breathing without assistance under the partial, but stable, mivacurium-induced paralysis, and intranasally administered neostigmine quickly relieved all clinically important muscular deficits despite insuring constant pressure on synaptic function by mivacurium because of its constant infusion rate.

FIG. 2 illustrates the time course of the experiment from the start of the mivacurium infusion (Time 0) to its termination at 135 min. Neurological deficits were stable within 100 min of the start of the mivacurium infusion and 15 min prior to neostigmine administration, whereas the stability of the neuromuscular blockade was established in the 10 min preceding neostigmine administration (105 min). Baseline visual acuity was 20/20 and became progressively worse until it exceeded 20/200 at the most advanced levels of neuromuscular blockade (FIG. 2A). Steady improvement was documented following neostigmine administration. In previously reported experiments, loss of visual acuity was one of the first deficits noted and last to recover with mivacurium infusion, which has been attributed to weakness of the extraocular muscles [43]. The ability to swallow was progressively impaired relatively early in the course of the experiment starting at about 80 min and recovered fully by 10 min after neostigmine administration (FIG. 2B).

FIG. 2C shows that the ability to lift the head (neck flexion) off the bed for more than 5 sec was lost at 100 min and fully recovered 5 min later by the first test of neck flexion following neostigmine administration.

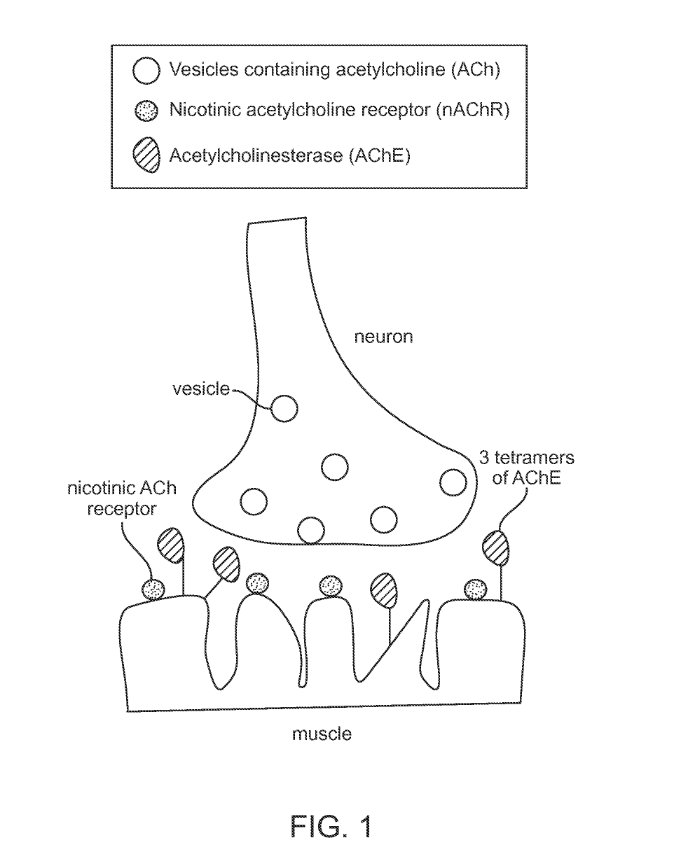

Peak flow (L/min) decreased from 100% of baseline to 72% of baseline (95% CI 64.72-78.61) and returned to an average of 91% of baseline after neostigmine administration (95% CI 85.24-97.26%) and was 95% of baseline by the termination of the mivacurium infusion (FIG. 1D). At first, the subject did not feel as if breathing was impaired, but at the deepest levels of neuromuscular blockade he experienced difficulty wrapping his lips around the peak flow meter and two measurements had to be repeated to guarantee no air leak.

Acceleromyographic and clinical assessments of adductor pollicis muscle function are summarized in Table 1. Briefly, the stimulating current was set 15 mA above threshold for the TOF device to detect thumb movement with final mAmps set at 39 mA based on a measured twitch threshold for the subject was 24 mA. The TOF ratio prior to neostigmine administration was stable at 0.56 and neostigmine was subsequently administered with the mivacurium infusion maintained at unchanged infusion rate of 2.5 ug/kg/min, enabling the investigators to determine the characteristics of the neostigmine effect. Neostigmine destabilized the adductor pollicis TOF ratio with preneostigmine with a peak improvement adductor pollicis TOF ratio of 0.70. Mean TOF ratios of 0.56±0.02, range 0.51-0.58, and CI 95% 0.54-0.57, and postneostigmine administration 0.64±0.03, range 0.61-0.70, and CI 95% (0.63-0.66).

Table 1 shows baseline clinical data in the first column compared to stable level of neuromuscular blockade by mivacurium as measured by adductor pollicis TOF (train-of-four) ratios and clinical impairment represented in the second (middle) column, and the third column shows the clinical response to intranasal neostigmine. Intranasal neostigmine antagonized the neuromuscular blockade as measured by TOF ratios and also improved all clinical levels of muscle function prior to termination of the mivacurium infusion. A constant rate of mivacurium infusion combined with stabilized TOF ratio and clinical impairment made it possible to compare changes attributable to the administration of intranasal neostigmine.

TABLE 1
RESPONSE TO INTRANASAL NEOSTIGMINE

| | Baseline | TOF ratio* | Response |
|---|---|---|---|
| Mivacurium Infu- | 0 | 2.5 mcg/kg/min | 2.5 mcg/kg/min |
| sion Rate | | | |
| Normalized TOF | 1.00 | 0.56 ± 0.02 | 0.64 ± 0.03 |
| ratios | | Range 0.51-0.58 | Range 0.61-0.70 |
| (mean ± SD) | | 95% CI | 95% CI |
| | | (0.54-0.57) | (0.63-0.66) |
| Visual Acuity | 20/20 | >20/200 | 20/20 |
| Ease of Swallowing | Easy | Difficult | Easy |
| Neck Flexion (Head | Easy | Unable | Easy |
| Raise >5 s) | (+) | (−) | (+) |
| Peak Flow | 100% | 72% | 91% |
| | | CI 95% | CI 95% |
| | | (64.72-78.61) | (85.24-97.26) |
| Jaw Ptosis | None | Present | None |
| Tongue Protrusion | Easy | Moderately | Easy |
| | | Difficult | |
| Diction | Normal | Poor | Improved |

*Stable Clinical Dysfunction + Stable Neuromuscular Blockade

There were no signs or symptoms of muscarinic-mediated toxicity while the mivacurium infusion was running, although the subject subsequently described the immediate and uncomfortable feeling of facial and lingual muscles “rearranging and tightening” within 5 min of receiving intranasal neostigmine. There was no stinging sensation or irritation or sense of swallowing the spray. There was no taste and no bronchospasm. No unanticipated effects occurred during the mivacurium infusion, but asymptomatic bradycardia was noted just prior to stopping the mivacurium infusion was easily reversed with IV glycopyrrolate, as were some symptoms concurrent with the episodes of bradycardia that included fasciculations and one brief episode of abdominal cramping. The observation of continued signs of neostigmine activity, including bradycardia, abdominal cramping, and fasciculations several hours after withdrawal of mivacurium suggested a long duration of neostigmine activity.

### REFERENCES CITED AND OF INTEREST

1 Jha, P., R. Kumar, A. Khera, M. Bhattacharya, P. Arora, V. Gajalakshmi. et al. 2010. HIV mortality and infection in India: estimates from nationally representative mortality survey of 1.1 million homes. Br. Med. J. 340:c621.
2 Girish, K. S., and K. Kemparaju. 2011. Overlooked issues of snakebite management: time for strategic approach. Curr. Top. Med. Chem. 11:2494-2508.
3 Mohapatra, B., D. A. Warrell, W. Suraweera, P. Bhatia, N. Dhingra, R. M. Jotkar, et al. 2011. Snakebite mortality in India: a nationally representative mortality survey. PLoS Negl. Trop. Dis. 5:e1018.
4 Simpson, I. D., and R. L. Norris. 2009. The global snakebite crisis-a public health issue misunderstood, not neglected. Wilderness Environ. Med. 20:43-56.
5 Kasturiratne, A., A. R. Wickremasinghe, N. de Silva, N. K. Gunawardena, A.

Pathmeswaran, R. Premaratna, et al. 2008. The global burden of snakebite: a literature analysis and modelling based on regional estimates of envenoming and deaths. PLoS Med. 5:e218.
6 Walker, M. B. 1935. Case showing the Effect of Prostigmin on Myasthenia Gravis. Proc. R. Soc. Med. 28:759-761.
7 Walker, M. B. 1938. Myasthenia Gravis: a Case in which Fatigue of the Forearm Muscles could induce Paralysis of the Extra-ocular Muscles. Proc. R. Soc. Med. 31:722.
8 Walker, M. 1946. The treatment of myasthenia gravis. Med. Press 216:81-84.
9 Johnston, J. D. 2007. Mary Broadfoot Walker (1888-1974). J. Neurol. 254:1306-1307.
10 Lingle, C. J., and J. H. Steinbach. 1988. Neuromuscular blocking agents. Int. Anesthesiol. Clin. 26:288-301.
11 Buzello, W., and C. Diefenbach. 1992. Curare and its successors. A 50-year's evolution. Anasthesiol Intensivmed Notfallmed Schmerzther 27:290-299.
12 Banerjee, R. N., A. L. Sahni, K. A. Chacko, and K. Vijay. 1972. Neostigmine in the treatment of Elapidae bites. J. Assoc. Physicians India 20:503-509.
13 Watt, G., R. D. Theakston, C. G. Hayes, M. L. Yambao, R. Sangalang, C. P. Ranoa, et al. 1986. Positive response to edrophonium in patients with neurotoxic envenoming by cobras (Naja naja philippinensis). A placebo-controlled study. N. Engl. J. Med. 315:1444-1448.
14 Currie, B., M. Fitzmaurice, and J. Oakley. 1988. Resolution of neurotoxicity with anticholinesterase therapy in death-adder envenomation. Med. J. Aust. 148:522-525.
15 WHO. 2010. Guidelines for the management of snake-bites in Southeast Asia. p. 106-107.
16 Warrell, D. A. 2012. Snake bite: a neglected problem in twenty-first century India. Natl Med. J. India 24:321-324.
17 WHO. 2010. Guidelines for the Prevention and Clinical Management of Snakebite in Africa. World Health Organization, Brazzaville, Congo, Pp. 87-88.
18 Fayrer, J. 1884. An Address on the Nature of Snake-Poison; its Effects on Living Creatures, and the Present Aspect of Treatment of the Poisoned. Br. Med. J. 1:205-210.
19 Hawgood, B. J. 1996. Sir Joseph Fayrer M D FRS (1824-1907) Indian Medical Service: snakebite and mortality in British India. Toxicon 34:171-182.
20 Drachman, D. B., D. M. Fambrough, and S. Satyamurti. 1973. Reduced acetylcholine receptors in myasthenia gravis. Trans. Am. Neurol. Assoc. 98:93-95.
21 Fambrough, D. M., D. B. Drachman, and S. Satyamurti. 1973. Neuromuscular junction in myasthenia gravis: decreased acetylcholine receptors. Science 182:293-295.
22 Satyamurti, S., D. B. Drachman, and F. Slone. 1975. Blockade of acetylcholine receptors: a model of myasthenia gravis. Science 187:955-957.
23 Drachman, D. B. 1978. Myasthenia gravis (second of two parts). N. Engl. J. Med. 298:186-193.
24 Drachman, D. B. 1978. Myasthenia gravis (first of two parts). N. Engl. J. Med. 298:136-142.
25 Flachsenberger, W., and P. Mirtschin. 1994. Anticholinesterases as antidotes to envenomation of rats by the death adder (Acanthophis antarcticus). Toxicon 32:35-39.
26 Guieu, R., J. P. Rosso, and H. Rochat. 1994. Anticholinesterases and experimental envenomation by Naja. Comp. Biochem. Physiol. C. Pharmacol. Toxicol. Endocrinol. 109:265-268.
27 Dash, S. C., S. K. Ghosh, D. C. Mathur, G. N. Jha, U. Prasad, and K. S. Grewal. 1976. Neurotoxic snake bite-dramatic recovery following neostigmine therapy. J. Assoc. Physicians India 24:535-537.
28 Sharma, S. K., P. Bovier, N. Jha, E. Alirol, L. Loutan, and F. Chappuis. 2013. Effectiveness of Rapid Transport of Victims and Community Health Education on Snake Bite Fatalities in Rural Nepal. Am. J. Trop. Med. Hyg. 00.
29 Fossati, A., M. G. Vimercati, G. L. Bandi, and A. Formenti. 1990. Pharmacokinetic study of neostigmine after intranasal and intravenous administration in the guinea pig. Drugs Exp. Clin. Res. 16:575-579.
30 Broggini, M., C. Benvenuti, V. Botta, A. Fossati, and M. Valenti. 1991. Bioavailability of intranasal neostigmine: comparison with intravenous route. Methods Find. Exp. Clin. Pharmacol. 13:193-198.
31 Ricciardi, R., B. Rossi, M. Nicora, A. Sghirlanzoni, and A. Muratorio. 1991. Acute treatment of myasthenia gravis with intranasal neostigmine: clinical and electromyographic evaluation. J. Neurol. Neurosurg. Psychiatry 54:1061-1062.
32 Dooley, J. M., K. J. Goulden, J. G. Gatien, E. J. Gibson, and B. S. Brown. 1986. Topical therapy for oropharyngeal symptoms of myasthenia gravis. Ann. Neurol. 19:192-194
33 Sghirlanzoni, A., D. Pareyson, C. Benvenuti, G. Cei, V. Cosi, M. Lombardi, et al. 1992. Efficacy of intranasal administration of neostigmine in myasthenic patients. J. Neurol. 239:165-169.
34 Mebs, D. 2002. Venomous and Poisonous Animals. CRC Press, Boca Raton, Fla. 339 p.
35 Sharma, S. K., F. Chappuis, N. Jha, P. A. Bovier, L. Loutan, and S. Koirala. 2004. Impact of snake bites and determinants of fatal outcomes in southeastern Nepal. Am. J. Trop. Med. Hyg. 71:234-238.
36 Alirol, E., S. K. Sharma, H. S. Bawaskar, U. Kuch, and F. Chappuis. 2010. Snake bite in South Asia: a review. PLoS Negl. Trop. Dis. 4:e603.
37 Sharma, S. K., S. Koirala, G. Dahal, and C. Sah. 2004. Clinico-epidemiological features of snakebite: a study from Eastern Nepal. Trop. Doct. 34:20-22.
38 Warrell, D. A., S. Looareesuwan, N. J. White, R. D. Theakston, M. J. Warrell, W. Kosakarn, et al. 1983. Severe neurotoxic envenoming by the Malayan krait Bungarus candidus (Linnaeus): response to antivenom and anticholinesterase. BMJ 286:678-680.
39 Anil, A., S. Singh, A. Bhalla, N. Sharma, R. Agarwal, and I. D. Simpson. 2010. Role of neostigmine and polyvalent antivenom in Indian common krait (Bungarus caeruleus) bite. J. Infect. Prev. 3:83-87.
40 Chiappinelli, V. 1996. K-neurotoxins and alpha-neurotoxins: Effect on neuronal nicotinic acetylcholine receptors. pp. 233-258 in A. Harvey, ed. Snake Toxins. Pergamon press, New York.
41 Singh, G., H. S. Pannu, P. S. Chawla, and S. Malhotra. 1999. Neuromuscular transmission failure due to common krait (Bungarus caeruleus) envenomation. Muscle. Nerve. 22:1637-1643.
42 Heier, T., J. E. Caldwell, M. L. Sharma, L. D. Gruenke, and R. D. Miller. 1994.

Mild intraoperative hypothermia does not change the pharmacodynamics (concentration-effect relationship) of vecuronium in humans. Anesth. Analg. 78:973-977.
43 Heier, T., J. E. Caldwell, J. R. Feiner, L. Liu, T. Ward, and P. M. Wright. 2010. Relationship between normalized adductor pollicis train-of-four ratio and manifestations of residual neuromuscular block: a study using acceleromyography during near steady-state concentrations of mivacurium. Anesthesiology 113:825-832.
44 Heier, T., J. R. Feiner, P. M. Wright, T. Ward, and J. E. Caldwell. 2012. Sex-related differences in the relationship between acceleromyographic adductor pollicis train-of-four ratio and clinical manifestations of residual neuromuscular block: a study in healthy volunteers during near steady-state infusion of mivacurium. Br. J. Anaesth. 108:444-451.
45 Di Costanzo, A., A. Toriello, C. Mannara, C. Benvenuti, and G. Tedeschi. 1993. Intranasal versus intravenous neostigmine in myasthenia gravis: assessment by computer analysis of saccadic eye movements. Clin. Neuropharmacol. 16:511-517.
46 Student's t-Test Calculator.
47 Brull, S. J., and G. S. Murphy. 2010. Residual neuromuscular block: lessons unlearned. Part II: methods to reduce the risk of residual weakness. Anesth. Analg. 111:129-140.
48 Curran, R. 2007. A milestone change in practice: a call for widespread application of intranasal medication delivery in the prehospital environment. Emerg. Med. Serv. 36: 40-41, 43-46, 48-49 passim.
49 Rajpal, S., G. Mittal, R. Sachdeva, M. Chhillar, R. Ali, S. S. Agrawal, et al. 2009. Development of atropine sulphate nasal drops and its pharmacokinetic and safety evaluation in healthy human volunteers. Environ. Toxicol. Pharmacol. 27:206-211.
50 Arora, P., S. Sharma, and S. Garg. 2002. Permeability issues in nasal drug delivery. Drug Discov. Today 7:967-975.
51 Chippaux, J. P. 2008. Estimating the global burden of snakebite can help to improve management. PLoS Med. 5:e221.

### 11.4 Example 4

### Intranasal Neostigmine Reduced Mortality from Experimental Naja Naja Envenomation in Mice

The effect of intranasal (IN) neostigmine, an acetylcholinesterase inhibitor (AchI) was investigated as a treatment for experimental envenomation by Naja naja (Indian cobra) in a murine model.

After pilot studies to assess the potency of reconstituted N. naja venom, 20 mice (21-28 g) were pseudo-randomized to receive IP injections of N. naja venom (approximately 2.5×LD50 for mice, Total N=20).

After 10 minutes, 15 animals received 5 microliters of 0.5 mg/mL neostigmine by nasal administration. Animals were observed continuously for 12 hours and assessed for signs of toxicity including respiratory distress, loss of spontaneous locomotor activity with the endpoint being death. Surviving animals were euthanized after 12 hours. Comparison of time to euthanasia across groups was performed and represented in Kaplan-Meier curves

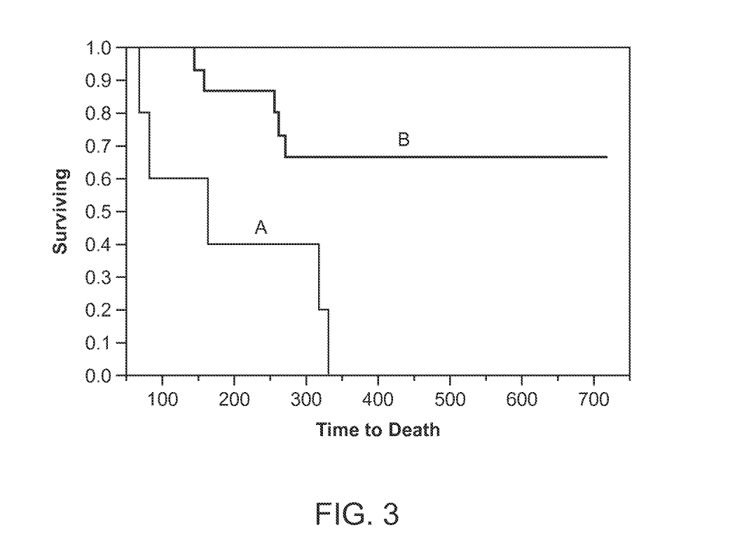

Results: As shown in FIG. 3, time to euthanasia for controls A) (venom alone, N=5) was 193 minutes (95% CI: 36-349). 5 of 5 controls died (100%) while B) 10 of 15 animals treated with IN neostigmine survived (67%) and were completely normal by 6 hours. Treatment with IN neostigmine (N=15) provided a significant increase in time to euthanasia was 553 minutes (95% CI: 415-689). FIG. 3: A) Venom alone (control) B) Venom+IN neostigmine.(treatment).

Swiss albino mice were envenomed with cobra toxin (Naja naja) by intraperitoneal injection at doses >2.5×the LD50 and that killed 100% of untreated mice “LD100”. Intranasal neostigmine was given 10 minutes after envenomation to determine if this intervention could delay or treat the envenomation. 10/15 mice recovered completely and were still behaving normally >12 hours after envenoming. 5/15 mice in the treatment group died compared to 5/5 (100%) of the envenomation group. Mice were treated only once, with no attempt to re-treat with intranasal neostigmine.

### Summary of Results (See FIG. 3):

5 uL of 0.5 mg/mL IN neostigmine saved the lives of 10/15 mice from 100% lethal dose of cobra (Naja naja)

5 uL of 0.5 mg/mL IN neostigmine prolonged or saved the lives of 5/5 mice given 2.5× the 100% lethal dose (“LD100”) of Naja naja venom.

5 uL of 0.5 mg/mL IN neostigmine trended toward prolonging the lives of mice given 5× the lethal dose of Naja naja.

### 11.5 Example 5

### Case Report

A healthy 50 year-old female was sleeping on a mud platform in her village and was awakened by a snake biting her left forearm. Within minutes, she developed headache and throat discomfort. She was transported by her family from the local clinic to the Emergency Department, conscious, but with progressive weakness. On physical examination, she had complete loss of gag reflex and rapidly worsening shortness of breath. There was no fang mark, ecchymosis or local swelling, and there were no other signs of coagulopathy such as bleeding gums or hematuria. She was unable to open her eyes and became unresponsive as emergent intubation was being performed without sedation. Polyvalent anti-snake venom (ASV) was administered concurrently. Based on the pattern of symptoms, lack of coagulopathy, normal renal function and normal bleeding and clotting times as well as lack of physical evidence of snakebite, krait bite (Bungarus sp.) was suspected [1,2]. Twenty-four hours after ASV therapy and continuous mechanical ventilation, she was awake and alert, but with residual weakness, including profound ptosis, bilateral 6th nerve palsies and inability to lift her neck. The persistent weakness despite an otherwise good recovery suggested that local application of an anticholinesterase might be of benefit. A single dose of 0.5 cc of aqueous 5% neostigmine was administered as an intranasal aerosol [3] with complete resolution of her ptosis within 30 minutes and almost complete recovery from her 6th nerve palsies. Intranasal neostigmine was administered every four hours thereafter with IV atropine as prophylaxis against cholinergic toxicity. She was extubated the following day and discharged from the hospital, with normal gait, station, speech, eye movements and respiratory efforts. At 2 week follow up she had returned to her usual activities, had good recall of her hospital course and noted having felt better almost immediately after receiving the nasal aerosol.

### REFERENCES CITED AND OF INTEREST

1. Warrell D A, Looareesuwan S, White N J, Theakston R D, Warrell M J, et al. (1983) Severe neurotoxic envenoming by the Malayan krait Bungarus candidus (Linnaeus): response to antivenom and anticholinesterase. Br Med J (Clin Res Ed) 286: 678-680.
2. Seneviratne U, Dissanayake S (2002) Neurological manifestations of snake bite in Sri Lanka. J Postgrad Med 48: 275-278; discussion 278-279.
3. Sghirlanzoni A, Pareyson D, Benvenuti C, Cei G, Cosi V, et al. (1992) Efficacy of intranasal administration of neostigmine in myasthenic patients. J Neurol 239: 165-169.
4. Theakston R D, Phillips R E, Warrell D A, Galagedera Y, Abeysekera D T, et al. (1990) Envenoming by the common krait (Bungarus caeruleus) and Sri Lankan cobra (Naja naja naja): efficacy and complications of therapy with Haffkine antivenom. Trans R Soc Trop Med Hyg 84: 301-308.
5. Anil A, Singh S, Bhalla A, Sharma N, Agarwal R, et al. (2010) Role of neostigmine and polyvalent antivenom in Indian common krait (Bungarus caeruleus) bite. J Infect Public Health 3: 83-87.
6. Warrell D A (2010) Guidelines for the management of snake-bites: South East Asia Region. New Delhi, India pp. 67.

The examples used in the specification are intended to help illustrate the invention, and are not intended to, nor should they be construed to, limit the scope of the invention. Variations of the invention, now known or further developed, are considered to fall within the scope of the present invention as described herein and as hereinafter claimed.

All publications and patent documents cited herein are incorporated herein by reference as if each such publication or document was specifically and individually indicated to be incorporated herein by reference. Citation of publications and patent documents is not intended as an indication that any such document is pertinent prior art, nor does it constitute any admission as to the contents or date of the same.

1. A method for treating or reducing the likelihood of neurotoxin-induced respiratory failure in a human subject, comprising determining that the subject is a victim of a snake bite and administering a pharmaceutically effective dose of an acetylcholinesterase inhibitor to the subject, wherein the administration is not via injection.

2. The method of claim 1 wherein determining that the subject is a victim of a snake bite comprises determining the subject is a victim of a venomous snake bite.

3. The method of claim 2 wherein the subject exhibits signs or symptoms of neurotoxic envenomation.

4. The method of claim 3 wherein the signs or symptoms of neurotoxic envenomation include one or more of ptosis, weakness of neck muscles, bulbar weakness, dysphagia, rnydriasis, fasiciculation, increased salivation, increased sweating, loss of muscle coordination, abdominal pain, difficulty speaking, nausea and vomiting, hypotension, respiratory distress and generalized and respiratory muscle paralyses.

5. The method of claim 1 wherein the subject does not exhibit symptoms of neurotoxic envenomation.

6. The method of claim 1, wherein a mAChR antagonist is not coadministered to the subject.

7. The method of claim 1, further comprising administering a pharmaceutically effective dose of an mAChR antagonist to the subject, wherein the administration is not by injection.

8. The method of claim 7 wherein the acetylcholinesterase inhibitor is administered prior to the mAChR antagonist.

9. The method of claim 7 wherein the mAChR antagonist is administered prior to the acetylcholinesterase inhibitor.

10. The method of claim 7 wherein the acetylcholinesterase inhibitor and the mAChR antagonist are administered within 5 minutes of each other.

11. The method of claim 7 wherein the acetylcholinesterase inhibitor and the mAChR antagonist are administered simultaneously as a mixture.

12. The method of claim 1 wherein the acetylcholinesterase inhibitor is intranasally administered.

13. The method of claim 1 wherein the acetylcholinesterase inhibitor is administered to the eye.

14. The method of claim 7 wherein the mAChR antagonist is intranasally administered.

15. The method of claim 7 wherein the mAChR antagonist is administered to the eye.

16. A method for treating neurotoxin-induced respiratory failure comprising:
identifying a victim who has been delivered a dose of venom by an animal and is suffering from clinical effects of envenomation, including early signs such as small muscle paralysis to deadly ones such as respiratory failure; and
intra-nasally or ocularly administering a pharmaceutically effective dose to the victim of an acetylcholinesterase; and
intra-nasally or ocularly administering a pharmaceutically effective dose to the victim of a mAChR antagonist and or acetylcholinesterase reactivating agent.

17. A method for treating or reducing the likelihood of residual neuromuscular blockade in a subject to whom a nondepolarizing neuromuscular blocking agent has been administered, the method comprising intra-nasally administering a pharmaceutically effective dose of an acetylcholinesterase inhibitor to the subject.

18. A method for treating or reducing the likelihood of residual neuromuscular blockade in a subject to whom a nondepolarizing neuromuscular blocking agent has been administered, the method comprising ocularly administering a pharmaceutically effective dose of an acetylcholinesterase inhibitor to the subject.

19. The method of claim 17 wherein the acetylcholinesterase inhibitor (AChl) is administered to the patient after completion of a medical procedure.

20. The method of claim 17 wherein the acetylcholinesterase inhibitor (AChl) is administered by mask or in line as an aerosol.

21. The method of claim 20 wherein the AChl is administered continuously for 1 to 30 minutes.

22. The method of claim 1 wherein the acetylcholinesterase inhibitor is a reversible acetylcholinesterase inhibitor.

23. The method of claim 22 wherein the acetylcholinesterase inhibitor is ambenonium; demarcarium; donepezil; edrophonium; galantamine; huperzine A; ladostigil; lactucopicrin; neostigmine; physostigmine; pyridostigmine; rivastigmine; tacrine; phospholine iodide; or ungeremine.

24. The method of claim 23 wherein the acetylcholinesterase inhibitor is edrophonium, neostigmine, physostigmine, pyridostigmine, tacrine; phospholine iodide; or ungeremine.

25. The method of claim 24 wherein the acetycholinesterase inhibitor is pyridostigmine.

26. The method of claim 7 wherein the mAChR antagonist is more selective for the mAChR than for the nAChR.

27. The method of claim 26 wherein the mAChR antagonist does not cross the blood brain barrier.

28. The method of claim 26 wherein the mAChR antagonist is atropine; benzatropine; glycopyrrolate; ipratropium; mebeverine; oxybutynin; pirenzepine; scopolamine; biperiden; tiotropium; or tropicamide.

29. The method of claim 28 wherein the mAChR antagonist is glycopyrrolate.

30. The method of any-preceding claim 1 wherein the subject is human.

31. A kit for use in treating envenomation comprising an acetycholinesterase inhibitor, a mAChR antagonist, and a drug delivery device.

32. A kit for use in treating envenomation comprising an acetycholinesterase inhibitor, a drug delivery device, and instructions for administration in response to envenomation.

33. A drug delivery device for managing neurotoxic envenoniation comprising a therapeutically effective dose of an acetycholinesterase inhibitor and a therapeutically effective dose of a mAChR antagonist.
